(19) United States
(12) Patent Application Publication
(10) Pub. No.: US 2023/0138092 A1
(43) Pub. Date: May 4, 2023

(54) Oral Care Compositions

(71) Applicant: Colgate-Palmolive Company, New York, NY (US)

(72) Inventors: Robert D'AMBROGIO, Princeton, NJ (US); Jean DENIS, Union, NJ (US)

(73) Assignee: Colgate-Palmolive Company, New York, NY (US)

(21) Appl. No.: 17/974,349

(22) Filed: Oct. 26, 2022

Related U.S. Application Data
(60) Provisional application No. 63/273,652, filed on Oct. 29, 2021.

(51) Int. Cl. A61K 8/27; A61K 8/21; A61K 8/46; A61Q 11/02

(52) U.S. Cl. CPC A61K 8/27; A61K 8/21; A61K 8/463; A61Q 11/02

(57) ABSTRACT

Described herein are oral care compositions comprising an effective amount of a stannous ion source and a taurate surfactant (e.g., sodium methyl cocoyl taurate). In one aspect the compositions of the disclosure can be used for the treatment or reduction of erosive tooth demineralization, gingivitis, plaque, and dental caries.

### FIELD

The present disclosure relates to oral care compositions comprising an effective amount of a stannous ion source and a taurate surfactant (e.g., sodium methyl cocoyl taurate). In one aspect the compositions of the disclosure can be used for the treatment or reduction of erosive tooth demineralization, gingivitis, plaque, and dental caries.

### BACKGROUND

Dental erosion involves demineralization and damage to the tooth structure due to acid attack from nonbacterial sources. Erosion is found initially in the enamel and, if unchecked, may proceed to the underlying dentin.

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar.

Stannous ion sources, such as stannous fluoride and stannous chloride, are known for use in clinical dentistry with a history of therapeutic benefits over forty years, and can have use in reducing certain bacterial growth in the oral cavity. However, until recently, the popularity of stannous ion sources has been limited by the instability in aqueous solutions. The instability of stannous salts in water is primarily due to the reactivity of the stannous ion (Sn²⁺). Stannous salts readily hydrolyze at a pH above 4, resulting in precipitation from solution. It has traditionally been thought that this formation of insoluble stannous salts results in a loss of therapeutic properties.

One common way to overcome the stability problems that can be associated with stannous ions is to limit the amount of water in the composition to very low levels, or to use a dual phase system. Both of these solutions to the stannous ion problem have drawbacks. Low water oral care compositions can be difficult to formulate with desired rheological properties, and dual-phase compositions are considerably more expensive to manufacture and package. Thus, it is preferable to formulate a high-water composition which uses an alternative means to maintain stable efficacious stannous ion concentrations.

Sodium lauryl sulfate (SLS) is widely used in dentifrice formulations surfactant. SLS has the benefits, for example, of being neutral with respect to product taste and often does not impact active ingredients stability. However, there has been recent consumer interest in developing various oral care products that do not contain sodium lauryl sulfate. For example, one of the concerns from using SLS has been the potential for skin or gum irritation. However, one of the drawbacks of developing formulations without SLS is that using new surfactant combinations in various oral care compositions (e.g., toothpaste) may lead to product separation because of the change of the ingredients balance in the formula. In some cases, a surfactant substitution—e.g., adding another surfactant to replace SLS—may potentially have a negative impact on the taste or active ingredients stability. Moreover, microbiological stability of the formulation can be negatively impacted by the absence of sodium lauryl sulfate. There are also production benefits to having SLS in a given formulation. For example, by removing SLS it may lead to a product being aerated during production and it may be more difficult to clean the equipment after the manufacturing process.

Thus, there is a need for providing improved stannous containing products for treating or preventing erosion of tooth enamel, that do not contain sodium lauryl sulfate, but nevertheless still have adequate stability, antimicrobial effectiveness and reduce plaque and treat or control gingivitis as traditional products that contain a sodium lauryl sulfate surfactant.

### BRIEF SUMMARY

In one aspect, compositions of the disclosure provide herein an oral care composition comprising:
A stannous ion source (e.g., stannous fluoride), and an effective amount of a taurate surfactant represented by Formula (1):

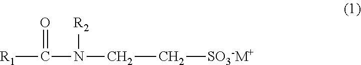

wherein R₁ is a saturated or unsaturated, straight or branched alkyl chain with 6 to 18 C atoms R₂ is H or methyl, and M⁺ is H, sodium, or potassium (e.g., sodium methyl cocoyl taurate).

Methods and uses for this composition are also described throughout. In at least one aspect, the compositions disclosed herein provide enhanced antibacterial activity compared to similar compositions that contain sodium lauryl sulfate. In some embodiments, the oral care composition is a toothpaste or oral gel composition.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight of the entire composition. The amounts given are based on the active weight of the material.

Compositions 1.0 et seq, which can include a toothpaste or oral gel, can comprise from 10% to 99% water, by weight of the composition. For example, the composition may comprise at least 10%, 15%, 20%, 25%, 30%, 35% or 40% water, up to a maximum of, for example, 60%, 70%, 80%, 90%, 95% or 99% water, by weight of the composition. As used herein, amounts of water refer to water added directly to the composition, as well as water added as part of ingredients or components which are added as aqueous solutions. In some embodiments, the composition comprises 10-60% water, or 10-50% water, or 10-40% water, or 10-30% water, or 15-30% water, or 20-30% water, about 25% water, about 30% water, or about 35% water by weight of the composition.

As used herein, the term “preformed salt”—e.g., when used in reference to zinc phosphate—means that the zinc phosphate is not formed in situ in the oral care composition, e.g., through the reaction of phosphoric acid and another zinc salt.

In one aspect, the present disclosure therefore provides an oral care composition (Composition 1.0) wherein the oral care composition comprises:
An effective amount of one or more stannous ion sources (e.g., stannous fluoride); and
An effective amount of a taurate surfactant, wherein the taurate surfactant is represented by Formula (1):

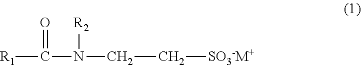

wherein R₁ is a saturated or unsaturated, straight or branched alkyl chain with 6 to 18 C atoms R₂ is H or methyl, and M⁺ is H, sodium, or potassium (e.g., sodium methyl cocoyl taurate).

For example, Composition 1.0 also includes the following:
1.1 Composition 1.0, wherein the R₁ is a saturated or unsaturated, straight or branched alkyl chain with 8 to 14 C atoms.
1.2 Composition 1.0 or 1.1, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations thereof.
1.3 Any of the preceding compositions, wherein the taurate surfactant comprises one or more surfactant selected from the group consisting of: sodium lauroyl methyl taurate (or sodium methyl lauroyl taurate), sodium methyl cocoyl taurate (SMCT), and combinations thereof.
1.4 Any of the preceding compositions, wherein the taurate surfactant comprises sodium methyl cocoyl taurate (e.g., 1%-5% by wt. of sodium methyl cocoyl taurate) (e.g., about 2% by wt. sodium methyl cocoyl taurate).
1.5 Any of the preceding compositions, wherein the taurate surfactant is present in an amount of from 0.25% to 5%, e.g., from 0.4% to 3%, e.g., from 0.4% to 2.75%, e.g., from 0.4% to 2.5%, e.g., from 0.5% to 3%, e.g., from 0.8% to 3%, e.g., from 1% to 3%, e.g., from 1.2% to 2.7%, e.g., from 1.5% to 3%, e.g., from 2% to 3%, e.g., from 1% to 2.8%, e.g., from 1% to 2.7%, e.g., from 1% to 2.5%, e.g., from 1.5% to 2.8%, e.g., from 1.5% to 2.5%, e.g., from 1.8% to 3%, e.g., from 1.8% to 2.8%, e.g., from 1.8% to 2.7%, e.g., from 1.8% to 2.5%, e.g., about 2% by weight of the composition.
1.6 Any of the preceding compositions, wherein the stannous ion source is selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations thereof.
1.7 Any of the preceding compositions, wherein the stannous ion source comprises stannous fluoride.
1.8 Any of the preceding compositions, wherein the stannous ion source comprises stannous fluoride in an amount of 0.1 wt. % to 2 wt. % (0.1 wt. %-0.6 wt. %) (e.g., about 0.454 wt. %) of the total composition weight.
1.9 Any of the preceding compositions, wherein the stannous ion source comprises stannous fluoride in an amount from 50 to 25,000 ppm (e.g., 750 -7000ppm, e.g., 1000-5000ppm, e.g., about 4500 ppm, e.g., about 4540ppm).
1.10 Any of the preceding compositions, wherein the composition comprises stannous fluoride and stannous pyrophosphate.
1.11 Any of the preceding compositions, wherein the composition comprises stannous fluoride and stannous chloride.
1.12 Any of the preceding compositions, wherein the one or more stannous ion source(s) is in an amount from 0.1%-5% by wt. of the total composition.
1.13 Any of the preceding compositions, wherein the composition comprises a zinc ion source and wherein the zinc ion source comprises one or more zinc salt(s) selected from the group consisting of: zinc citrate, zinc oxide, zinc phosphate, zinc lactate, zinc sulfate, zinc silicate, zinc gluconate and combinations thereof.
1.14 Any of the preceding compositions, wherein the composition comprises a zinc ion source and wherein the zinc ion source comprises zinc oxide.
1.15 Any of the preceding compositions, wherein the composition comprises a zinc ion source and wherein the zinc ion source comprises zinc citrate.
1.16 Any of the preceding compositions, wherein the composition comprises a zinc ion source and wherein the zinc ion source comprises zinc oxide and zinc citrate.
1.17 Any of the preceding compositions, wherein the ratio of the amount of zinc oxide (e.g., wt. %) to zinc citrate (e.g., wt. %) is from 1.5:1 to 4.5:1 (e.g., 2:1, 2.5:1, 3:1, 3.5:1, or 4:1).
1.18 Any of the preceding compositions, wherein the zinc citrate is in an amount of from 0.25 to 0.75 wt. % (e.g., 0.5 wt. %) and zinc oxide may be present in an amount of from 0.75 to 1.25 wt. % (e.g., 1.0 wt. %) based on the weight of the oral care composition.
1.19 Any of the preceding compositions wherein the zinc citrate is about 0.5 wt. %.
1.20 Any of the preceding compositions wherein the zinc oxide is about 1.0 wt. %.
1.21 Any of the preceding compositions where the zinc citrate is about 0.5 wt. % and the zinc oxide is about 1.0 wt. %.
1.22 Any of the preceding compositions, wherein the composition comprises a zinc ion source and wherein the zinc ion source comprises zinc phosphate (e.g., wherein the zinc phosphate is a preformed salt of zinc phosphate) (e.g., zinc phosphate hydrate) (e.g., from 0.5-4wt % of zinc phosphate) (e.g., about 1.0 wt. % of zinc phosphate).
1.23 The preceding composition, wherein the zinc phosphate is added as a pre-formed salt.
1.24 Any of the preceding compositions, wherein the composition comprises a source of zinc ions, and wherein the zinc ion source comprises zinc lactate.
1.25 Any of the preceding compositions, wherein the zinc ion source is in an amount from 0.1%-5% by wt. of the total composition (e.g., zinc phosphate from 0.1%-5% by wt. of the total composition).
1.26 Any preceding composition comprising an effective amount of a fluoride ion source.
1.27 The preceding composition, wherein the amount of the fluoride ion source is in an amount from 0.01% to 5% by weight, relative to the weight of the oral care composition, for example, from 0.05 to 4% by weight, or from 0.1% to 3% by weight, or from 0.2 to 2% by weight, or from 0.3 to 1% by weight, or from 0.3 to 0.5% by weight, or about 0.32% by weight (e.g., 0.32% by weight).
1.28 Any of the preceding compositions, wherein the fluoride source is selected from the group consisting of: sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides and combinations thereof.
1.29 The preceding composition, wherein the fluoride ion source comprises sodium fluoride (e.g., from 0.2%-2% by wt. of sodium fluoride)
1.30 The composition of 1.28, wherein the fluoride ion source comprises stannous fluoride (e.g., stannous fluoride from 0.1%-2% by wt. of the total composition).
1.31 The composition of 1.28, wherein the fluoride ion source comprises sodium monofluorophosphate.
1.32 Any preceding composition, wherein the composition comprises water in the amount of 10% by weight or more, relative to the weight of the oral care composition, for example, 10-90%, or 10-80%, or 10-70%, or 10-60%, or 10-50%, or 10-40%, or 10-30%, or 15-30%, 15% -40% 20% -40%, 20-35%, or 20-50%, or 30-35%, or about 25% or about 30%, by weight of the composition.
1.33 Any preceding composition, further comprising an organic buffer system, wherein the buffer system comprises a carboxylic acid and one or more conjugate base salts thereof, for example, alkali metal salts thereof (e.g., citric acid and sodium citrate).
1.34 Any preceding composition, wherein the composition comprises the organic acid buffer system in an amount of 0.1 to 5.0% by weight of the composition, measured as the combined amount of organic acid and any conjugate base salts (e.g., citric acid and sodium citrate); for example, from 0.5 to 4.0%, or from 1.0 to 3.0%, or from 1.5 to 3.0%, or from 1.0 to 2.4%, or from 1.0% to 2.0%, or from 1.0% to 1.5%, or about 1.2%, by weight of the composition.
1.35 Any preceding composition, wherein the oral care composition further comprises an abrasive, for example, silica abrasives, calcium abrasives, and other abrasives as disclosed herein.
1.36 Any preceding composition, further comprising one or more humectants, as described herein, e.g., selected from sorbitol, glycerol, xylitol and propylene glycol, or combinations thereof, e.g., a combination of sorbitol and glycerin.
1.37 Any of the preceding compositions, wherein the zwitterionic surfactant comprises cocamidopropyl betaine, (e.g., in an amount of 0.1-5% by weight) (e.g., about 0.6% by wt.).
1.38 Any preceding composition, further comprising an effective amount of one or more alkali phosphate salts for example orthophosphates, pyrophosphates, tripolyphosphates, tetraphosphates or higher polyphosphates.
1.39 The preceding composition, wherein the alkali phosphate salts comprise tetrasodium pyrophosphate or tetrapotassium pyrophosphate, for example, in an amount of 0.5 to 5% by weight of the composition, e.g., 1-4%, or about 2-4%, or about 1-2% or about 1.5% or about 2% or about 4%, by weight.
1.40 The preceding composition, wherein the alkali phosphate salts comprise sodium tripolyphosphate or potassium tripolyphosphate, for example, in an amount of 0.5 to 6% by weight of the composition, e.g., 1-4%, or 2-3% or about 3% by weight.
1.41 Any preceding composition, further comprising a whitening agent.
1.42 Any preceding composition, wherein the oral care composition is in the form selected from: dentifrice (e.g., a toothpaste or oral gel), powder (e.g., tooth powder), cream, mouthwash, strip or gum (e.g., chewing gum).
1.43 Any preceding composition, wherein the pH of the composition is from 6 to 9, such as from 6.5 to 8, or from 6.5 to 7.5, or about 7.0.
1.44 Any preceding composition, wherein the composition is a single-phase composition (e.g., not a dual-phase composition).
1.45 Any preceding composition, wherein the composition is essentially free or free of phosphates of more than four phosphate groups.
1.46 Any preceding composition, wherein the composition is essentially free or free of phosphates of more than three phosphate groups.
1.47 Any preceding composition, wherein the composition is essentially free or free of hexametaphosphate salts (e.g., sodium hexametaphosphate).
1.48 Any of the preceding compositions, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.
1.49 Any preceding compositions, wherein the composition further comprises a polymer selected from the group consisting of: carboxymethyl cellulose (free form or a salt, e.g., sodium salt), a gum (e.g., xanthan gum, carrageenan gum, or gum arabic), polyethylene glycol (e.g., polyethylene glycol 200, 400, 600 or 800, or a mixture thereof), and a combinations thereof, for example, a mixture of sodium carboxy methyl cellulose, xanthan gum, polyethylene glycol 600.
1.50 Composition 1.49, wherein the polymer comprises sodium carboxy methyl cellulose.
1.51 Composition 1.49, wherein the polymer comprises xanthan gum.
1.52 Any preceding composition further comprising a silica thickener and/or a silica abrasive.
1.53 Any preceding composition, wherein the oral care composition comprises an additional anionic surfactant that is not sodium lauryl sulfate, wherein the additional anionic surfactant is selected from the group consisting of: water-soluble salts of higher fatty acid monoglyceride monosulfates (such as the sodium salt of the monosulfated monoglyceride of hydrogenated coconut oil fatty acids such as sodium N-methyl N-cocoyl taurate), sodium cocomonoglyceride sulfate, higher alkyl-ether sulfates (e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na)), higher alkyl aryl sulfonates such as sodium dodecyl benzene sulfonate (sodium lauryl benzene sulfonate), higher alkyl sulfoacetates (such as sodium lauryl sulfoacetate (dodecyl sodium sulfoacetate)), higher fatty acid esters of 1,2 dihydroxy propane sulfonate, sulfocolaurate (N-2-ethyl laurate potassium sulfoacetamide) and sodium lauryl sarcosinate.
1.54 Any of the preceding compositions wherein the composition comprises an amino acid.
1.55 The preceding composition wherein the amino acid is a basic amino acid (e.g., arginine)
1.56 Any of the preceding compositions wherein the amino acid is a basic amino acid provided in the form of a di- or tri-peptide comprising arginine or lysine, or salts thereof.
1.57 Any of the preceding compositions wherein the basic amino acid comprises arginine or lysine, and wherein the arginine or lysine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.
1.58 Any of the preceding compositions wherein the amino acid comprises arginine from 0.1 wt. %-6.0 wt. % (e.g., about 1.5 wt. %) (e.g., about 5 wt. %) of the total composition, wherein the weight of the arginine is calculated as free form.
1.59 Any of the preceding compositions wherein the amino acid is arginine from about 1.5 wt. of the total composition, wherein the weight of the arginine is calculated as free form.
1.60 Any of the preceding compositions wherein the amino acid is arginine from 4.5 wt. %-8.5 wt. % (e.g., about 5.0 wt. %) of the total composition, wherein the weight of the basic amino acid is calculated as free form.
1.61 Any of the preceding compositions wherein the amino acid is arginine from about 5.0 wt. % of the total composition, wherein the weight of the basic amino acid is calculated as free form.
1.62 Any of the preceding compositions wherein the amino acid is arginine from 3.5 wt. %-9 wt. % of the total composition, wherein the weight of the basic amino acid is calculated as free form.
1.63 Any of the preceding compositions wherein the amino acid is L-arginine.
1.64 Any of the preceding compositions wherein the amino acid is a free form arginine.
1.65 Any of the preceding compositions wherein the amino acid is arginine or lysine in partially or wholly in salt form.
1.66 Composition 1.65 wherein the amino acid is arginine phosphate.
1.67 Composition 1.65 wherein the amino acid is arginine hydrochloride.
1.68 Composition 1.65 wherein the amino acid is arginine bicarbonate.
1.69 Any of the preceding compositions wherein the amino acid is arginine or lysine ionized by neutralization with an acid or a salt of an acid.
1.70 Any foregoing composition comprising a zwitterionic surfactant.
1.71 The preceding composition, wherein the zwitterionic surfactant is a betaine zwitterionic surfactant (e.g., from 0.1%-5% by wt. of the total composition) (e.g., 0.2%-1% by wt. of the total composition) (e.g., about 0.6% by wt. of the total composition).
1.72 The preceding composition, wherein the betaine zwitterionic surfactant is a C8-C16 aminopropyl betaine (e.g., cocamidopropyl betaine).
1.73 The preceding composition wherein the C8-C16 aminopropyl betaine is cocamidopropyl betaine.
1.74 The preceding composition wherein the cocamidopropyl betaine, is present in an amount of from 0.5% to 4% by wt. of the total composition.
1.75 The preceding composition, wherein the cocamidopropyl betaine is from 0.1% to 3% by wt. of the total composition.
1.76 The preceding composition wherein the cocamidopropyl betaine is from 0.1% to 1% (e.g., about 0.6% by wt. of the total composition).
1.77 Any of the preceding compositions wherein the composition comprises cocamidopropyl betaine and sodium methyl cocoyl taurate in a wt. % ratio of (e.g., wt. %) is from 0.1:1 to 1:1 (e.g., 0.1:1, 0.2:1, 0.3:1, 0.4:1 or 0.5:1) (e.g., 0.3:1)
1.78 Any preceding composition, wherein the oral care composition is free of sodium lauryl sulfate.
1.79 Any preceding composition, wherein the composition comprises:
   Zinc phosphate;
   Stannous fluoride;
   Sodium methyl cocoyl taurate; and
   An orally acceptable carrier.
1.80 Any preceding composition, wherein the composition comprises:
   Zinc phosphate;
   Stannous fluoride;
   Arginine;
   Sodium methyl cocoyl taurate; and
   An orally acceptable carrier.
1.81 Any preceding composition, wherein the composition comprises:
   Stannous fluoride;
   Zinc citrate and/or zinc lactate and/or zinc oxide (e.g., zinc lactate) (e.g., zinc citrate and zinc oxide);
   Sodium methyl cocoyl taurate; and
   An orally acceptable carrier.
1.82 Any preceding composition, wherein the composition comprises:
   Zinc oxide;
   Zinc citrate;
   Stannous fluoride;
   Sodium methyl cocoyl taurate; and
   An orally acceptable carrier.
1.83 Any preceding composition, wherein the composition comprises:
   Stannous fluoride;
   Stannous chloride;
   Zinc citrate or zinc lactate;
   Sodium methyl cocoyl taurate; and
   An orally acceptable carrier.
1.84 Any preceding composition, wherein the composition comprises:
   Stannous fluoride;
   Stannous chloride;
   One or more zinc salt(s) selected from: zinc oxide, zinc citrate, zinc lactate and combinations thereof; (e.g., zinc lactate) (e.g., zinc citrate and zinc oxide) (e.g., zinc citrate and zinc lactate) (e.g., zinc lactate and zinc oxide)
   Sodium methyl cocoyl taurate; and
   An orally acceptable carrier.
1.85 Any preceding composition, wherein the composition comprises:
   Zinc phosphate from 0.5% -4% by wt. of the composition;
   Stannous fluoride from 0.1-2% by wt. of the composition;
   Sodium methyl cocoyl taurate from 0.5-5% by wt. of the composition; and
   An orally acceptable carrier.
1.86 Any preceding composition, wherein the composition comprises:
   Stannous fluoride from 0.1-2% by wt. of the composition;
   Zinc citrate from 0.25%-0.75% by wt. of the composition;
   Sodium methyl cocoyl taurate from 0.5-5% by wt. of the composition; and
   An orally acceptable carrier.
1.87 Any preceding composition, wherein the composition comprises:
   Zinc oxide from 0.5%-1.5% by wt. of the composition;
   Zinc citrate from 0.25%-0.75% by wt. of the composition;
   Stannous fluoride from 0.1-2% by wt. of the composition;
   Sodium methyl cocoyl taurate from 0.5-5% by wt. of the composition; and
   An orally acceptable carrier.
1.88 Any preceding composition, wherein the composition comprises:
   Stannous fluoride from 0.1-2% by wt. of the composition;
   Stannous chloride from 0.1-2% by wt. of the composition;
   Zinc citrate from 0.1%-2% by wt. of the composition;
   Sodium methyl cocoyl taurate from 0.5-5% by wt. of the composition; and
   An orally acceptable carrier.
1.89 Any preceding composition, wherein the composition comprises:
   Stannous fluoride from 0.1-2% by wt. of the composition;
   Stannous chloride from 0.1-2% by wt. of the composition;
   One or more zinc salts in an amount from 0.5%-5%, wherein the zinc salt(s) is selected from: zinc oxide, zinc citrate, zinc lactate and combinations thereof; (e.g., zinc lactate) (e.g., zinc citrate and zinc oxide) (e.g., zinc citrate and zinc lactate) (e.g., zinc lactate and zinc oxide)
   Sodium methyl cocoyl taurate from 0.5-5% by wt. of the composition; and
   An orally acceptable carrier.
1.90 Any preceding composition, wherein the composition comprises:
   Zinc phosphate from 0.5% -4% by wt. of the composition;
   Stannous fluoride from 0.1-2% by wt. of the composition;
   Sodium methyl cocoyl taurate from 0.5-5% by wt. of the composition;
   Arginine from 0.5-10% by wt. of the composition (e.g., about 1.5% by wt.) (e.g., about 5% by wt.), wherein the amount of arginine is calculated in free form; and
   An orally acceptable carrier.
1.91 Any preceding composition, wherein the composition comprises:
   Zinc phosphate from 0.5% -4% by wt. of the composition;
   Stannous fluoride from 0.1-2% by wt. of the composition;
   Stannous pyrophosphate from 0.1%-2% by wt. of the composition;
   Sodium methyl cocoyl taurate from 0.5-5% by wt. of the composition; and
   An orally acceptable carrier.
1.92 Any of the compositions of 1.79-1.91 further comprising cocamidopropyl betaine, in an amount of from 0.1% to 5% by wt. of the total composition.
1.93 Any preceding composition wherein the composition does not contain any sodium lauryl sulfate.
1.94 Any of composition 1.0-1.92 wherein the composition is substantially free of sodium lauryl sulfate.
1.95 Any of the preceding compositions, wherein the oral care composition is a dentifrice (e.g., a toothpaste or oral gel), powder (e.g., tooth powder), cream, mouthwash, strip or gum (e.g., chewing gum).
1.96 Any of the preceding compositions further comprising a preservative selected from: benzyl alcohol, Methylisothizolinone (“MIT”), Sodium bicarbonate, lauryl alcohol, and polyphosphate.
1.97 Any of the preceding compositions comprising nitric acid or a water-soluble nitrate salt (e.g., potassium nitrate).
1.98 The preceding composition, wherein the water-soluble nitrate salt is selected from an alkali or alkaline earth metal nitrate, or zinc nitrate, silver nitrate, or ammonium nitrate.
1.99 The preceding composition, wherein the water-soluble nitrate salt is an alkali metal nitrate salt or an alkaline earth metal nitrate salt.
1.100 The preceding composition, wherein the nitrate salt is selected from lithium nitrate, sodium nitrate, potassium nitrate, magnesium nitrate, and calcium nitrate.
1.101 The preceding composition, wherein the nitrate salt is potassium nitrate.
1.102 Any preceding composition, wherein the oral care composition is free or substantially free of sodium lauryl sulfate

The compositions may optionally comprise additional ingredients suitable for use in oral care compositions. The compositions of Composition 1.0 et seq may be formulated in a suitable dentifrice base, e.g., comprising abrasives, e.g., silica abrasives, surfactants, foaming agents, vitamins, polymers, enzymes, humectants, thickeners, additional antimicrobial agents, preservatives, flavorings, colorings, and/or combinations thereof. Examples of suitable dentifrice bases are known in the art. Alternatively, the compositions may be formulated as a gel (e.g., for use in a tray), chewing gum, lozenge or mint. Examples of suitable additional ingredients that can be employed in the compositions of the present disclosure are discussed in more detail below.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

Active Agents: The compositions of the disclosure may comprise various other agents that are active to protect and enhance the strength and integrity of the enamel and tooth structure and/or to reduce bacteria and associated tooth decay and/or gum disease or to provide other desired benefits. Effective concentration of the active ingredients used herein will depend on the particular agent and the delivery system used. The concentration will also depend on the exact salt or polymer selected. For example, where the active agent is provided in salt form, the counterion will affect the weight of the salt, so that if the counterion is heavier, more salt by weight will be required to provide the same concentration of active ion in the final product.

Compositions of the disclosure may contain from 0.1 to 1 wt. % of an antibacterial agent, such as about 0.3 wt. %. Any suitable antimicrobial actives can be employed.

Fluoride Ion Source: The oral care compositions of any of Composition 1.0 et seq can include one or more additional fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al, the disclosure of each of which is hereby incorporated by reference in their entirety. Representative fluoride ion sources include, but are not limited to, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes sodium fluoride, sodium monofluorophosphate as well as mixtures thereof. In certain embodiments, the oral care composition of the disclosure may contain stannous fluoride and any additional source of fluoride ions or fluorine-providing agents in amounts sufficient to supply, in total, from 25 ppm to 25,000 ppm (mass fraction) of fluoride ions, generally at least 500 ppm, e.g., from 500 to 2000 ppm, e.g., from 1000 to 1600 ppm, e.g., about 1450 ppm. The appropriate level of fluoride will depend on the particular application. A toothpaste for general consumer use would typically have from1000 to about 1500 ppm, with pediatric toothpaste having somewhat less. A dentifrice or coating for professional application could have as much as 5,000 or even about 25,000 ppm fluoride. Additional fluoride ion sources may be added to the compositions of the disclosure at a level of from 0.01 wt. % to 10 wt. % in one embodiment or from 0.03 wt. % to 5 wt. %, and in another embodiment from 0.1 wt. % to 1 wt. % by weight of the composition. As discussed above, weights of fluoride salts to provide the appropriate level of fluoride ion will vary based on the weight of the counterion in the salt.

Abrasives: The compositions of any of Composition 1.0 et seq can include abrasives. Examples of suitable abrasives include silica abrasives, such as standard cleaning silicas, high cleaning silicas or any other suitable abrasive silicas. Additional examples of abrasives that can be used in addition to or in place of the silica abrasives include, for example, a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄ • 2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, or combinations thereof.

Silica abrasive polishing materials useful herein, as well as the other abrasives, generally have an average particle size ranging between 0.1 and 30 microns, such as between 5 and 15 microns. The silica abrasives can be from precipitated silica or silica gels, such as the silica xerogels described in U.S. Pat. No. 3,538,230, to Pader et al. and U.S. Pat. No. 3,862,307, to Digiulio, the disclosures of which are incorporated herein by reference in their entireties. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, the disclosure of which is incorporated herein by reference in its entirety. In certain embodiments, abrasive materials useful in the practice of the oral care compositions in accordance with the disclosure include silica gels and precipitated amorphous silica having an oil absorption value of less than 100 cc/100 g silica, such as from 45 cc/100 g to 70 cc/100 g silica. Oil absorption values are measured using the ASTA Rub-Out Method D281. In certain embodiments, the silicas are colloidal particles having an average particle size of from 3 microns to 12 microns, and from 5 to 10 microns. Examples of low oil absorption silica abrasives useful in the practice of the disclosure are marketed under the trade designation Sylodent XWA® by Davison Chemical Division of W. R. Grace & Co., Baltimore, Md. 21203. Sylodent 650 XWA®, a silica hydrogel composed of particles of colloidal silica having a water content of 29% by weight averaging from 7 to 10 microns in diameter, and an oil absorption of less than 70 cc/100 g of silica is an example of a low oil absorption silica abrasive useful in the practice of the present disclosure.

Any suitable amount of silica abrasive can be employed. Examples of suitable amounts include 10 wt. % or more dry weight of silica particles, such as from 15 wt. % to 30 wt. % or from 15 wt. % to 25 wt. %, based on the total weight of the composition.

Foaming agents: The oral care compositions of any of Composition 1.0 et seq, may include an agent to increase the amount of foam that is produced when the oral cavity is brushed. Illustrative examples of agents that increase the amount of foam include, but are not limited to polyoxyethylene and certain polymers including, but not limited to, alginate polymers. The polyoxyethylene may increase the amount of foam and the thickness of the foam generated by the oral care compositions of the present disclosure. Polyoxyethylene is also commonly known as polyethylene glycol (“PEG”) or polyethylene oxide. The polyoxyethylenes suitable for compositions of the present disclosure may have a molecular weight of from 200,000 to 7,000,000. In one embodiment the molecular weight may be from 600,000 to 2,000,000 and in another embodiment from 800,000 to 1,000,000. Polyox® is the trade name for the high molecular weight polyoxyethylene produced by Union Carbide. The foaming agent, (e.g., polyoxyethylene) may be present in an amount of from 0.1% to 50%, in one embodiment from 0.5% to 20% and in another embodiment from 1% to 10%, or from 2% to 5% by weight of the oral care compositions of the present disclosure.

Surfactants: The compositions of any of Composition 1.0 et seq may comprise an anionic surfactant that is not sodium lauryl sulfate. For example, in one aspect, any of Composition 1.0 et seq can additionally comprise any of the following surfactants:
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na),
iii. higher alkyl aryl sulfonates such as sodium dodecyl benzene sulfonate (sodium lauryl benzene sulfonate),
iv. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

By “higher alkyl” is meant, e.g., C₆₋₃₀ alkyl. In certain embodiments, the anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauroyl sarcosinate and sodium coconut monoglyceride sulfonates are examples of anionic surfactants of this type. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. In one embodiment, the anionic surfactant is present in a toothpaste at from 0.3% to 4.5% by weight, e.g., about 1.5%. The compositions of the disclosure may optionally contain mixtures of surfactants, e.g., comprising anionic surfactants and other surfactants that may be anionic, cationic, zwitterionic or nonionic. Generally, suitable surfactants are those which are reasonably stable throughout a wide pH range. Surfactants are described more fully, for example, in U.S. Pat. No. 3,959,458, to Agricola et al.; U.S. Pat. No. 3,937,807, to Haefele; and U.S. Pat. No. 4,051,234, to Gieske et al, the disclosures of which are incorporated herein by reference in their entireties.

The surfactant or mixtures of compatible surfactants that are included in addition to the anionic surfactants can be present in the compositions of the present disclosure in from 0.1% to 5.0%, in another embodiment from 0.3% to 3.0% and in another embodiment from 0.5% to 2.0% by weight of the total composition. These ranges do not include the anionic surfactant amounts.

The compositions of any of Composition 1.0 et seq include a zwitterionic surfactant, for example a betaine surfactant, for example cocamidopropylbetaine, e.g., in an amount of from 0.1% to 4.5% by weight, e.g., from 0.5 to 2% by weight cocamidopropylbetaine.

Tartar control agents: In various embodiments of the present disclosure, the compositions of any of Composition 1.0 et seq can comprise an anticalculus (tartar control) agent. Suitable anticalculus agents include, without limitation, phosphates and polyphosphates (for example pyrophosphates and tripolyphosphates), polyaminopropanesulfonic acid (AMPS), hexametaphosphate salts, zinc citrate trihydrate, polypeptides, polyolefin sulfonates, polyolefin phosphates, and diphosphonates. The compositions of the disclosure thus may comprise phosphate salts in addition to the zinc phosphate. In particular embodiments, these salts are alkali phosphate salts, e.g., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; and dimeric phosphates such as pyrophosphates; and multimeric phosphates, such as tripolyphosphates, tetraphosphates, hexaphosphates and hexametaphosphates (e.g., sodium hexametaphosphate). In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions may comprise tetrasodium pyrophosphate in an amount of from 0.5 to 5% by weight, e.g., 1-3%, or 1-4%, or 2-4%, or 1-2% or about 2%, or about 4% by weight of the composition. In another embodiment, the compositions may comprise a mixture of tetrasodium pyrophosphate (TSPP) and sodium tripolyphosphate (STPP), e.g., in proportions of TSPP at from 0.5 to 5 wt. %, such as from 1 to 2 wt. % or 1 to 4 wt. % and STPP at from 0.5% to 6 wt. %, such as 1 to 4%, or 2 to 3% by weight of the composition. Such phosphates are provided in an amount effective to reduce erosion of the enamel, to aid in cleaning the teeth, and/or to reduce tartar buildup on the teeth, for example in an amount of from 0.2 to 20 wt. %, e.g., from 1 to 15 wt. %, by weight of the composition.

Flavoring Agents: The oral care compositions of any of Composition 1.0 et seq may also include a flavoring agent. Flavoring agents which are used in the practice of the present disclosure include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. The flavoring agent may be incorporated in the oral composition at a concentration of from 0.1 to 5% by weight e.g., from 0.5 to 1.5% by weight.

Polymers: The oral care compositions of any of Composition 1.0 et seq may also include additional polymers to adjust the viscosity of the formulation or enhance the solubility of other ingredients. Such additional polymers include polyethylene glycols, polysaccharides (e.g., cellulose derivatives, for example carboxymethyl cellulose, hydroxymethyl cellulose, ethyl cellulose, microcrystalline cellulose or polysaccharide gums, for example xanthan gum, guar gum or carrageenan gum). Acidic polymers, for example polyacrylate gels, may be provided in the form of their free acids or partially or fully neutralized water-soluble alkali metal (e.g., potassium and sodium) or ammonium salts. In one embodiment, the oral care composition may contain PVP. PVP generally refers to a polymer containing vinylpyrrolidone (also referred to as N-vinylpyrrolidone, N-vinyl-2-pyrrolidone and N-vinyl-2-pyrrolidinone) as a monomeric unit. The monomeric unit consists of a polar imide group, four non-polar methylene groups and a non-polar methane group.

In some embodiments, the compositions of the disclosure comprise one or more polyethylene glycols, for example, polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

Silica thickeners, which form polymeric structures or gels in aqueous media, may be present. Note that these silica thickeners are physically and functionally distinct from the particulate silica abrasives also present in the compositions, as the silica thickeners are very finely divided and provide little or no abrasive action. Other thickening agents are carboxyvinyl polymers, carrageenan, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate can also be used as a component of the thickening composition to further improve the composition's texture. In certain embodiments, thickening agents in an amount of from 0.5% to 5.0% by weight of the total composition are used.

In some embodiments, the compositions of any of Composition 1.0 et seq may include an anionic polymer, for example in an amount of from 0.05 to 5%. Examples of such agents generally known for use in dentifrice are disclosed in U.S. Pat. Nos. 5,188,821 and 5,192,531, both of which are incorporated herein by reference in their entirety; and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (M.W.) of from 30,000 to 1,000,000, such as from 300,000 to 800,000. These copolymers are available for example as Gantrez, e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805. The enhancing agents when present are present in amounts ranging from 0.05 to 3% by weight. Other operative polymers include those such as the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2-methylpropane sulfonic acid having a molecular weight of from 1,000 to 2,000,000. Another useful class of polymeric agents includes polyamino acids containing proportions of anionic surface-active amino acids such as aspartic acid, glutamic acid and phosphoserine, e.g., as disclosed in U.S. Pat. No. 4,866,161, issued to Sikes et al., which is also incorporated herein by reference in its entirety.

In some embodiments, there are no anionic polymers present in the composition. In other embodiments, there may be anionic polymers present, but they do not include copolymers of methyl vinyl ether and maleic acid or anhydride.

Humectants: Within certain embodiments of any of Composition 1.0 et seq, it is also desirable to incorporate a humectant to prevent the composition from hardening upon exposure to air. Certain humectants can also impart desirable sweetness or flavor to dentifrice compositions. Suitable humectants include edible polyhydric alcohols such as glycerin, sorbitol, xylitol, propylene glycol as well as other polyols and mixtures of these humectants. In one embodiment of the disclosure, the principal humectant is one of glycerin, sorbitol or a combination thereof. The humectant may be present at levels of greater than 15 wt. %, such as from 15 wt. % to 55 wt. %, or from 20 wt. % to 50 wt. %, or from 20 wt. % to 40 wt. %, or about 20% or about 30% or about 40%, based on the total weight of the composition.

Amino Acids: In some aspects, Compositions 1.0 et seq can comprise a basic amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrulline, ornithine, creatine, histidine, diaminobutanoic acid, diaminoproprionic acid, salts thereof or combinations thereof. In a particular embodiment, the basic amino acids are selected from arginine, citrulline, and ornithine. In certain embodiments, the basic amino acid is arginine, for example, L-arginine, or a salt thereof.

In another aspect, the compositions of the invention (e.g., Compositions 1.0 et seq) can further comprise one or more neutral amino acid, which can include, but is not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

Other optional ingredients: In addition to the above-described components, any of Composition 1.0 et seq can contain a variety of optional oral care ingredients some of which are described below. Optional ingredients include, for example, but are not limited to, adhesives, sudsing agents, flavoring agents, sweetening agents such as sodium saccharin, additional antiplaque agents, abrasives, aesthetics such as TiO₂ coated mica or other coloring agents, such as dyes and/or pigments.

In some embodiments, the compositions of the present disclosure can have any pH suitable for in a product for use in oral care. Examples of suitable pH ranges are from 6 to 9, such as from 6.5 to 8, or 6.5 to 7.5, or about 7.0.

In some embodiments, the oral care compositions of any of Composition 1.0 et seq are either essentially free of, free of, or do not include any sodium hexametaphosphate. In some embodiments, the oral care compositions of the present disclosure are either essentially free of, free of, or do not include any halogenated diphenyl ethers (e.g., triclosan).

In some aspects the oral care compositions of any of Composition 1.0 et seq are either essentially free of, free of, or do not include any sodium lauryl sulfate.

By “essentially free” it is meant that the compositions have no more than 0.01% by weight of these compounds.

In some embodiments, the compositions of the present disclosure are either essentially free of, free of or do not include any complexing agents for increasing solubility of zinc phosphate. Examples of known complexing agents that can be excluded from the compositions of the present disclosure include the chelating agents taught in U.S. Patent Application No. 2007/0025928, the disclosure of which is hereby incorporated by reference in its entirety. Such chelating agents include mineral surface-active agents, including mineral surface-active agents that are polymeric and/or polyelectrolytes and that are selected from phosphorylated polymers, wherein if the phosphorylated polymer is a polyphosphate, the polyphosphate has average chain length of 3.5 or more, such as 4 or more; polyphosphonates; polycarboxylates; carboxy-substituted polymers; copolymers of phosphate- or phosphonate-containing monomers or polymers with ethylenically unsaturated monomers, amino acids, proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); and mixtures thereof. Other known complexing agents that can be excluded from the compositions of the present disclosure include those taught in CA 2634758, the disclosure of which is incorporated here by reference in its entirety. Examples include polyphosphorylated inositol compounds such as phytic acid, myo-inositol pentakis(dihydrogen phosphate); myo-inositol tetrakis(dihydrogen phosphate), myo-inositol trikis(dihydrogen phosphate), and alkali metal, alkaline earth metal or ammonium salts of any of the above inositol compounds. Phytic acid is also known as myo-inositol 1,2,3,4,5,6-hexakis (dihydrogen phosphate) or inositol hexaphosphoric acid.

In some embodiments, the compositions of the disclosure (e.g., any of Composition 1.0 et seq) can comprise a non-ionic block copolymer, optionally together with an alkyl glucoside. The non-ionic block copolymer may be a poly(propylene oxide)/poly(ethylene oxide) copolymer. In some embodiments, the copolymer has a polyoxypropylene molecular mass of from 3000 to 5000 g/mol and a polyoxyethylene content of from 60 to 80 mol %. In some embodiments, the non-ionic block copolymer is a poloxamer. In some embodiments, the non-ionic block copolymer is selected from: Poloxamer 338, Poloxamer 407, Poloxamer, 237, Poloxamer, 217, Poloxamer 124, Poloxamer 184, Poloxamer 185, and a combination of two or more thereof. In some embodiments, the copolymer is Poloxamer 407. In some embodiments, the compositions of the disclosure (e.g., any of Composition 1.0 et seq) can comprise a betaine amphoteric surfactant and a non-ionic block copolymer, optionally together with an alkyl glucoside.

The compositions of the invention (e.g., Composition 1.0 et seq) are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

In another aspect, the present disclosure provides a method of treatment or prevention of erosive tooth demineralization, repair of enamel, gingivitis, plaque, and/or dental caries, the method comprising the application to the oral cavity of a person in need thereof a composition according to the invention (e.g., Composition 1.0 et seq), e.g., by brushing, for example, one or more times per day.

In another aspect, the present disclosure provides a method for reducing erosion of an enamel surface comprising preparing an oral care composition according to the invention (e.g., Composition 1.0 et seq), e.g., and applying the composition to the enamel surface, for example by brushing.

In another aspect, the present disclosure provides a method of using the compositions described herein (e.g., any of Compositions 1.0 et seq) to treat, reduce or control the incidence of enamel erosion. The methods comprise applying any of the compositions as described herein to the teeth, e.g., by brushing, or otherwise administering the compositions to the oral cavity of a subject in need thereof. The compositions can be administered regularly, such as, for example, one or more times per day. In various embodiments, administering the compositions of the present disclosure to a patient can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) increase relative levels of non-cariogenic and/or non-plaque forming bacteria, (ix) reduce or inhibit formation of dental caries, (x) reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; (xv) reduce tartar build-up, and/or (xvi) promote systemic health, including cardiovascular health, e.g., by reducing potential for systemic infection via the oral tissues. The disclosure further provides compositions for use in any of the above methods. Further embodiments provide methods wherein at least one tooth is remineralized after administration of a composition as described herein.

In yet another aspect, the compositions disclosed herein (e.g., any of Compositions 1.0 et seq) provide improved repair of acid softened enamel. For example the compositions disclosed herein (e.g., any of Compositions 1.0 et seq) can be administered, to a subject in need thereof, as part of a method to increase strength or hardness of damaged and/or softened enamel. In another aspect, the compositions disclosed herein (e.g., any of Compositions 1.0 et seq) can be administered, to a subject in need thereof, as part of a method to promote remineralization of enamel.

The present application further discloses a method of making any of the compositions of the present disclosure, e.g., any of Composition 1.0 et seq.

### EXAMPLES

### Example 1—Dentifrice Formulation

In one aspect, representative Dentifrice Formulations according to the present disclosure are prepared according to Table 1 below:

TABLE 1
Table 1. Examples of Stannous Fluoride Toothpastes Containing SLS vs Taurate Surfactant

| | FORMULA | | | | | |
|---|---|---|---|---|---|---|
| | A | B | C | D | E | F |
| Ingredients (by wt %.) | Wt % | Wt % | Wt % | Wt % | Wt % | Wt % |
| SORBITOL - NON-CRYSTAL - 70% | 39.0 | 39.0 | 31.6 | 30.4 | 36.7 | 35.4 |
| SOLN USP, EP | | | | | | |
| GLYCERIN | 6.0 | 6.0 | 6.0 | 6.0 | 8.0 | 8.0 |
| ABRASIVE | 20.0 | 20.0 | 20.0 | 20.0 | 20.0 | 20.0 |
| THICKENER | 2.3 | 2.3 | 2.3 | 2.3 | 2.05 | 2.05 |
| DEMINERALIZED WATER | q.s. | q.s. | q.s. | q.s. | q.s. | q.s. |
| POLYMER | 3.3 | 3.3 | 3.3 | 3.3 | 3.3 | 3.3 |
| TETRASODIUM PYROPHOSPHATE - | 2.0 | 2.0 | 2.5 | 2.5 | 2.5 | 2.5 |
| FINE FCC | | | | | | |
| TRISODIUM CITRATE DIHYDRATE - | 1.0 | 1.0 | 1.6 | 1.6 | 1.5 | 1.5 |
| USP | | | | | | |
| FLAVOR, COLORANT AND | 2.54 | 2.54 | 2.54 | 2.54 | 2.54 | 2.54 |
| SWEETENER | | | | | | |
| STANNOUS PYROPHOSPHATE | | | | | 1.0 | 1.0 |
| L-ARGININE | | | 1.3 | 1.3 | | |
| COCAMIDOPROPYL BETAINE (30% | 1.25 | 2.0 | 1.25 | 2.0 | 1.25 | 2.0 |
| SOL'N) | | | | | | |
| GANTREZ S-97 (B.F.) - 16.8% SOLN | | | 6.0 | 6.0 | | |
| CITRIC ACID - ANHYDROUS USP, EP | 0.2 | 0.2 | 0.1 | 0.1 | 0.2 | 0.2 |
| ZINC PHOSPHATE HYDRATE | 1.0 | 1.0 | 1.0 | 1.0 | 1.0 | 1.0 |
| STANNOUS FLUORIDE, USP | 0.454 | 0.454 | 0.454 | 0.454 | 0.454 | 0.454 |
| SODIUM LAURYL SULFATE | 1.5 | | 1.5 | | 1.5 | |
| POWDER | | | | | | |
| SODIUM METHYL COCOYL | | 2.0 | | 2.0 | | 2.0 |
| TAURATE | | | | | | |
| Total Components | 100.0 | 100.0 | 100.0 | 100.0 | 100.0 | 100.0 |

### Example 2—Stability

Below is a summary of the stability of stannous fluoride toothpastes (described in Table 1) at 4, 8 and 13 weeks.

TABLE 2

| | | A | B | C | D | E | F |
|---|---|---|---|---|---|---|---|
| pH (10% Soln) | Initial | 7.2 | 7.0 | 7.3 | 7.2 | 6.8 | 6.8 |
| | 4 wks-40 C./75% RH | 7.3 | 7.0 | 7.2 | 7.3 | 6.8 | 6.9 |
| | 8 wks-40 C./75% RH | 7.2 | 7.1 | 7.3 | 7.2 | 7.0 | 6.9 |
| | 13 wks-30 C./65% RH | 7.2 | 7.0 | 7.3 | 7.2 | 6.9 | 6.9 |
| | 13 wks-40 C./75% RH | 7.3 | 7.2 | 7.3 | 7.3 | 6.9 | 7.0 |
| Sol. Fluoride | Initial | 1120 | 1072 | 1100 | 1113 | 1048 | 1122 |
| (ppm) | 4 wks-40 C./75% RH | 1030 | 1024 | 1043 | 1086 | 1005 | 1010 |
| | 8 wks-40 C./75% RH | 1011 | 1003 | 1014 | 1037 | 964 | 996 |
| | 13 wks-30 C./65% RH | 1050 | 1034 | 981 | 997 | 980 | 983 |
| | 13 wks-40 C./75% RH | 990 | 979 | 974 | 966 | 892 | 922 |
| Total Tin (%) | Initial | 0.36 | 0.36 | 0.36 | 0.36 | 0.97 | 0.95 |
| Sol. Tin (%) | Initial | 0.27 | 0.28 | 0.27 | 0.28 | 0.86 | 0.89 |
| | 4 wks-40 C./75% RH | 0.22 | 0.24 | 0.23 | 0.24 | 0.88 | 0.79 |
| | 8 wks-40 C./75% RH | 0.21 | 0.24 | 0.21 | 0.23 | 0.83 | 0.75 |
| | 13 wks-30 C./65% RH | 0.23 | 0.24 | 0.22 | 0.24 | 0.83 | 0.78 |
| | 13 wks-40 C./75% RH | 0.21 | 0.24 | 0.21 | 0.22 | 0.76 | 0.73 |

The formulas in Table 1 are evaluated for chemical and physical stability per ICH accelerated aging/stress guidelines. Table 2 indicates that these formulas are sufficiently stable for fluoride and are acceptably buffered to maintain pH within 6.5-7.5 target range. Regarding soluble tin, formulas B & D—both containing sodium methyl cocoyl taurate—appear to provide increased soluble tin values over the high temperature/stress conditions as compared to Formulas A and C—both which contain sodium lauryl sulfate. Formulas E and F—which contain relatively higher amounts of tin as compared to Formulas A-D—show similar soluble fluoride results over the 3-month accelerated aging period. Here, Table 2 demonstrates that formulations with both sodium methyl cocoyl taurate and SLS are acceptably stability for pH, soluble fluoride (ppm) and soluble tin (%) under accelerated shelf-life conditions.

### Example 3—Stannous Uptake

Metal Uptake on Hard and Soft Tissues: The formulations described in Table 1 are measured for metal uptake utilizing representative hard and soft tissue substrates with bovine enamel & Vitroskin assays, respectively. These established in vitro methods demonstrate that formulas with sodium methyl cocoyl taurate (B, D & F) had either directionally or statistically greater tin uptake compared to comparable toothpaste formulas with sodium lauryl sulfate surfactant (A, C & E) as summarized in tables 3 & 4.

TABLE 3
Tin Uptake on Hard Tissue

| | | Statistical |
|---|---|---|
| Sample | Avg μg/cm² | Comparison* |
| Formula A | 1.20 +/− 0.10 | A |
| Formula B | 1.37 +/− 0.08 | AB |
| Formula C | 1.39 +/− 0.06 | B |
| Formula D | 1.58 +/− 0.05 | C |
| Formula E | 2.46 +/− 0.10 | D |
| Formula F | 2.63 +/− 0.11 | D |

*Means that don't share common letter = Sign. Diff @95% CI, Tukey method, N = 3 per cell

TABLE 4
Tin Uptake on Soft Tissue

| | | Statistical |
|---|---|---|
| Sample | Avg μg/cm² | Comparison* |
| Formula A | 2.10 +/− 0.12 | A |
| Formula B | 2.39 +/− 0.09 | B |
| Formula C | 2.43 +/− 0.12 | B |
| Formula D | 2.94 +/− 0.09 | C |
| Formula E | 4.10 +/− 0.14 | D |
| Formula F | 4.31 +/− 0.16 | D |

*Means that don't share common letter = Sign. Diff @95% CI, Tukey method, N = 3 per cell

### Example 4—Antibacterial Effect

Formulas A & B are further evaluated by in vitro methods to determine antibacterial performance. Two key in vitro tests are University of Manchester model and Plaque Glycolysis model which are described below.

University of Manchester Model: The anaerobic model (UoM) is used to provide a more sensitive indication of potential efficacy of the formula. In this model, saliva is collected from 4 healthy volunteers and pooled together for use as inoculum. Each sample is treated in triplicate twice a day for 8 days. Biofilm is recovered after 16 treatments to measure for ATP (RLU) as an end point for viable bacteria. Toothpastes demonstrating lower APT scores provide more effective antibacterial performance. Finally, in UoM studies, a commercial toothpaste containing NaF and KNO3 is used as the negative control.

In this test, Formula B (containing sodium methyl cocoyl taurate) surprisingly demonstrates a statistically significantly improvement at controlling anaerobic biofilm as compared to a comparable formula that contains sodium lauryl sulfate as the surfactant in lieu of sodium methyl cocoyl taurate (Formula A). Both Formula A and Formula B demonstrate statistically significant improvement relative to the negative control.

TABLE 5
Viable Bacteria as ATP (RLU) - Manchester Model, Test 1

| | | Statistical |
|---|---|---|
| Sample | Avg Log RLU | Comparison* |
| Commercial toothpaste (negative | 4.94 +/− 0.22 | A |
| control)** | | |
| Stannous Fluoride (formula A) | 4.24 +/− 0.13 | B |
| Stannous Fluoride (formula B) | 3.97 +/− 0.12 | C |

*Means that don't share common letter = Sign. Diff @95% CI, Tukey method, N = 26 per cell
**Negative Control, NaF, KNO3 formula

Plaque glycolysis Model: Studies indirectly measuring biofilm health involve an in vitro adaptation of the Plaque Glycolysis Model discussed in Donald J. White, et. al., Journal of Clinical Dentistry, #6 Special Issue, Pp 69-78, 1995, the contents of which are herein incorporated by reference. Briefly, the method quantifies the glycolytic effects of toothpaste formulas on treated in vitro biofilm pool of both anaerobic and aerobic bacteria. The efficacy of each toothpaste formula is based on biofilm pH change. A lower average pH change indicates reduction of viable bacteria and greater antibacterial performance of the respective test toothpaste. Finally, a commercial toothpaste containing NaF and KNO3 actives is used as the negative control.

In this test, Formula B (sodium methyl cocoyl taurate surfactant) was equally effective at reducing a general oral bacteria population compared to Formula A (sodium lauryl sulfate surfactant). Both Formula A and Formula B perform significantly better than the negative control (a regular sodium fluoride toothpaste) at controlling the bacterial biofilm.

TABLE 6
Plaque Glycolysis Study - Average
pH Change with Treatment, Test 1

| | | Statistical |
|---|---|---|
| Sample | Avg pH Change | Comparison* |
| Commercial toothpaste (negative | 2.02 +/− 0.10 | A |
| control)** | | |
| Stannous Fluoride (formula A) | 1.32 +/− 0.04 | B |
| Stannous Fluoride (formula B) | 1.35 +/− 0.04 | B |

*Means that don't share common letter = Sign. Diff @95% CI, Tukey method, N = 3 per cell
**Negative Control, NaF, KNO3 formula

### Example 4—Acid Challenge Study

A 5-day cycling study using an automated robotic system was performed determine the relative erosion protection potential of toothpastes containing stannous fluoride or sodium fluoride as a fluoride source, and either sodium lauryl sulfate or sodium methyl cocoyl taurate as surfactant, compared to control toothpaste containing sodium lauryl sulfate surfactant and lacking fluoride. The dentifrices were evaluated for their ability to prevent enamel loss and demineralization upon repeated acid challenge on an enamel substrate. Microhardness was used as a before and after marker for erosion protection. Quantification of calcium and phosphate release by an acid solution was investigated by a colorimetric method [Attin et al., 2005] to determine the extent of enamel demineralization. See Attin T. et al. Method to detect minimal amounts of calcium dissolved in acidic solutions; Caries Res 2005a; 39:432-436; and Attin T. et al., Suitability of a malachite green procedure to detect minimal amounts of phosphate dissolved in acidic solutions. Clin Oral Investig. 2005b; 9:203-207.

Disk Creation

This study used 2 disks per dentifrice/Formula, with each disk comprising 10 bovine cores affixed to each disk. 100 3mm bovine rods were cut down to the appropriate size to form the cores. The rods (cores) come with an excess of clear acrylic that is cut off close to the point where white acrylic meets clear acrylic using a Buehler Stone Cutting blade. The rods are then placed on the disks and sanded down and polished. The enamel sides of the rods are sanded down and polished (using a EcoMet 250 Grinder Polisher) in stages, making sure that at all stages the disks are flush and even. While grinding, water is flowing over the surfaces, and a small amount of fabuloso soap can be put on the circulating pad to give a little bit less friction. The cores are polished with 320, 400, 800 and then 1200 grit, using the following parameters:

Time=2 minutes

Platen Speed=180 rpm, Head Speed=60 rpm

Fluids=Fresh Water

Head Rotation=Contra Rotation

Mode=Single

Force=1 psi

Once a desired surface was reached, the cores were polished with 6 um Yellow MetaDi Diamond suspension polishing fluid on a white Trident polishing pad using the following parameters:

Time=6 minutes

Platen Speed=180 rpm, Head Speed=60 rpm

Fluids=Off

Head Rotation=Contra Rotation

Mode=Single

Force=2 psi

At 2 minutes elapsed time additional 6um Yellow MetaDi Diamond suspension polishing fluid was added onto rotating platen. At 4 minutes elapsed time 6 gms of 0.05 μm white MasterPrep Alumina polishing fluid was added onto rotating platen.

Microhardness Readings

Microhardness readings were taken to quantify the efficacy of the tested Formulas against enamel erosion. The readings were taken before a 5% Citric Acid, post etch, and then after the pH cycling (Demin/Remin). A MicroMet 6020 microindentation hardness tester running Omnimet software was used to take hardness readings. The instrument makes an indent in the shape of a diamond (⋄). The width from the left most point to the right most point is measured. This measurement is known as the hardness amount and is given in HK values. For each bovine core, 3 measurements were made and were in the middle of the core.

5% Citric Acid Etch

The disks were etched in a 5% Citric Acid solution before the pH cycling began. A 200 ml solution of 5% Citric Acid was made and then poured into a beaker. Each disk was individually placed in a 5% Citric Acid solution for 30 seconds, making sure each disk was fully submersed. After the 30 seconds the disks were washed in Deionized water and then patted dry with a Kim-wipe. Once all the disks were treated, they were measured again on the MicroMet6020, and robotic cycling was commenced.

5 Day Robotic pH Cyling

The following steps 1-27 were programmed and run robotically in the following order:

| | | | |
|---|---|---|---|
| 1 | Rinse | 6 | Seconds |
| 2 | Saliva | 1 | hour |
| 3 | Rinse | 6 | seconds |
| 4 | Treatment (Toothpaste) | 2 | minutes |
| 5 | Rinse | 3 | seconds |
| 6 | Saliva | 1 | hour |
| 7 | Rinse | 6 | seconds |
| 8 | Acid Challenge | 2 | minutes |
| 9 | Rinse | 3 | seconds |
| 10 | Saliva | 1 | hour |
| 11 | Rinse | 6 | seconds |
| 12 | Acid Challenge | 2 | minutes |
| 13 | Rinse | 3 | seconds |
| 14 | Saliva | 1 | hour |
| 15 | Rinse | 6 | seconds |
| 16 | Acid Challenge | 2 | minutes |
| 17 | Rinse | 3 | seconds |
| 18 | Saliva | 1 | hour |
| 19 | Rinse | 6 | seconds |
| 20 | Acid Challenge | 2 | minutes |
| 21 | Rinse | 6 | seconds |
| 22 | Saliva | 1 | hour |
| 23 | Rinse | 6 | seconds |
| 24 | Treatment | 2 | minutes |
| 25 | Rinse | 3 | seconds |
| 26 | Saliva | 16-18 | hours |
| 27 | Rinse | 6 | seconds |

The following compositions shown in Table 6 were prepared and tested:

TABLE 6
Toothpaste Formulations Tested in pH Cycling Study

| | Formula | | | |
|---|---|---|---|---|
| | A | B | C | D |
| Ingredients (wt %) | Wt % | Wt % | Wt % | Wt % |
| HYDRATED SILICA | 21.500 | 21.500 | 21.500 | 21.500 |
| DEMINERALIZED WATER | 18.000 | 18.000 | 18.000 | 18.000 |
| PEG-12 | 2.000 | 2.000 | 2.000 | 2.000 |
| TETRASODIUM | 2.000 | 2.000 | 2.000 | 2.000 |
| PYROPHOSPHATE | | | | |
| SODIUM LAURYL SULFATE | 1.500 | 1.500 | 1.500 | 0.000 |
| POWDER | | | | |
| SODIUM METHYL COCOYL | 0.000 | 0.000 | 0.000 | 2.000 |
| TAURATE | | | | |
| COCAMIDOPROPYL BETAINE | 1.250 | 1.250 | 1.250 | 2.000 |
| TRISODIUM CITRATE | 0.600 | 0.600 | 1.000 | 1.000 |
| DIHYDRATE - USP | | | | |
| CITRIC ACID - ANHYDROUS | 0.600 | 0.600 | 0.200 | 0.200 |
| USP, EP | | | | |
| FLAVOR | 1.000 | 1.000 | 1.000 | 1.000 |
| Microcrystalline Cellulose/ | 1.000 | 1.000 | 1.000 | 1.000 |
| Sodium CMC NF | | | | |
| ZINC PHOSPHATE | 1.000 | 1.000 | 1.000 | 1.000 |
| MONOHYDRATE | | | | |
| SODIUM SACCHARIN USP or | 0.400 | 0.400 | 0.400 | 0.400 |
| EP | | | | |
| TITANIUM DIOXIDE - USP, EP | 0.500 | 0.500 | 0.500 | 0.500 |
| STANNOUS FLUORIDE, USP | 0.000 | 0.000 | 0.454 | 0.454 |
| SODIUM FLUORIDE, USP | 0.000 | 0.243 | 0.000 | 0.000 |
| SODIUM CMC | 0.800 | 0.800 | 0.800 | 0.800 |
| XANTHAN GUM | 0.300 | 0.300 | 0.300 | 0.300 |
| SUCRALOSE USP, EP | 0.010 | 0.010 | 0.010 | 0.010 |
| GLYCERIN - USP, EP VEG | 6.000 | 6.000 | 6.000 | 6.000 |
| SORBITOL - 70% SOLN USP, EP | q.s. | q.s. | q.s. | q.s. |
| Total Components | 100.000 | 100.000 | 100.000 | 100.000 |
| Formula pH (10% Soln in water) | 7.42 | 7.46 | 7.28 | 7.33 |

Note:
ratio of citric acid/trisodium citrate buffers was adjusted as needed to achieve similar pH for all samples.

Results & Discussion

In this pH cycling test model there were 20 runs (n=20) completed for each treatment. The two stannous fluoride toothpastes described in Table 6 were compared to a control toothpaste without fluoride and also to a toothpaste containing the same 1100 ppm amount of fluoride but from NaF instead of SnF₂. Microhardness measurements using 50 g force for 5 seconds (KHN) were made with each test sample. The percent difference between baseline and after pH cycling were then calculated. Colorimetric evaluations were also used to determine the % enamel loss/demineralization. These results are summarized in Table 7 below.

Toothpastes providing a lower value have more effectively protected the enamel cores. As expected, the fluoride-free toothpaste was less effective in preventing acid erosion while the fluoride-containing formulations were overall more effective in maintaining enamel hardness and preventing demineralization. However, it was unexpectedly found that the stannous fluoride toothpaste with taurate surfactant (D) provided both 1) improved enamel protection compared to the SLS-containing toothpaste that otherwise shared a common formulation (C); and also 2) improved protection against demineralization. The taurate formulation was also previously shown to provide improved metal uptake to model oral surfaces (compared to SLS) and thus appears to provide a more effective mineral shield against acid attack.

TABLE 7
Enamel Protection Capabilities Of Toothpastes
(From Table 1) with Acid Challenges

| | % Change in Microhardness (KHN) | |
|---|---|---|
| Sample | Post pH Cycling vs Core Baseline | % Demineralization |
| A | 62.10 ± 3.84^{A} | 65.21 ± 4.73^{A} |
| B | 46.52 ± 4.14^{B} | 46.15 ± 2.46^{B} |
| C | 38.76 ± 2.41^{C} | 41.22 ± 2.11^{C} |
| D | 33.43 ± 2.12^{D} | 33.90 ± 1.71^{D} |

Note:
Statistical Evaluations Use the Tukey Method and 95% Confidence. Means that do not share a letter are significantly different.

1. An oral care composition comprising:
An effective amount of one or more stannous ion sources; and
An effective amount of a taurate surfactant, wherein the taurate surfactant is represented by Formula (1): 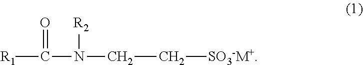
wherein R₁ is a saturated or unsaturated, straight or branched alkyl chain with 6 to 18 C atoms R₂ is H or methyl, and M⁺ is H, sodium, or potassium; for example wherein R₁ is a saturated or unsaturated, straight or branched alkyl chain with 8 to 14 C atoms.

2. (canceled)

3. The oral care composition of claim 1, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, and combinations thereof.

4. The oral care composition of claim 1, wherein the taurate surfactant comprises sodium methyl cocoyl taurate.

5. The oral care composition of claim 1, wherein the stannous ion source is selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations thereof.

6. The oral care composition of claim 1, wherein the stannous ion source comprises stannous fluoride; or stannous fluoride and stannous chloride; or stannous fluoride and stannous pyrophosphate.

7. (canceled)

8. (canceled)

9. The oral care composition of claim 1, wherein the one or more stannous ion source(s) is in an amount from 0.1%-5% by wt. of the total composition.

10. The oral care composition of claim 1, wherein the composition comprises a zinc ion source and wherein the zinc ion source comprises one or more zinc salt(s) selected from the group consisting of: zinc citrate, zinc oxide, zinc phosphate, zinc lactate, zinc sulfate, zinc silicate, zinc gluconate and combinations thereof.

11. The oral care composition of claim 1, wherein the composition comprises a zinc ion source and wherein:
the zinc ion source comprises zinc oxide; or
the zinc ion source comprises zinc citrate; or
the zinc ion source comprises zinc phosphate.

12. (canceled)

13. The oral care composition of claim 11, wherein the zinc citrate is in an amount of from 0.25 to 0.75 wt % and zinc oxide may be present in an amount of from 0.75 to 1.25 wt % based on the weight of the oral care composition.

14. (canceled)

15. The oral care composition of claim 1, wherein the composition comprises an amino acid; for example wherein the amino acid is a basic amino acid; for example wherein the basic amino acid comprises arginine or lysine, and wherein the arginine or lysine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.

16. (canceled)

17. (canceled)

18. The oral care composition of claim 1 comprising a zwitterionic surfactant; for example wherein the zwitterionic surfactant is a betaine zwitterionic surfactant; for example wherein the betaine zwitterionic surfactant is a C8-C16 aminopropyl betaine (e.g., cocamidopropyl betaine).

19. (canceled)

20. (canceled)

21. (canceled)

22. The oral care composition of claim 1, wherein the composition comprises:
Zinc phosphate;
Stannous fluoride;
Sodium methyl cocoyl taurate; and
An orally acceptable carrier;
or the composition comprises:
Zinc phosphate;
Stannous fluoride;
Arginine;
Sodium methyl cocoyl taurate; and
An orally acceptable carrier.

23. (canceled)

24. The oral care composition of claim 1, wherein the composition comprises:
Stannous fluoride;
Zinc citrate or zinc lactate;
Sodium methyl cocoyl taurate; and
An orally acceptable carrier;
or the composition comprises:
Zinc oxide;
Zinc citrate;
Stannous fluoride;
Sodium methyl cocoyl taurate; and
An orally acceptable carrier.

25. (canceled)

26. The oral care composition of claim 1, wherein the composition comprises:
Stannous fluoride;
Stannous chloride;
Zinc citrate;
Sodium methyl cocoyl taurate; and
An orally acceptable carrier.

27. The oral care composition of claim 1, wherein the composition comprises:
Stannous fluoride;
Stannous pyrophosphate;
Zinc phosphate;
Sodium methyl cocoyl taurate; and
An orally acceptable carrier.

28. An oral care composition of claim 1, wherein the composition is free or substantially free of sodium lauryl sulfate.

29. An oral care composition of claim 1, wherein the oral care composition is a dentifrice, powder, cream, mouthwash, strip or gum.

30. A method of treatment or prevention of erosive tooth demineralization, gingivitis, plaque, and/or dental caries, the method comprising the application to the oral cavity of a person in need thereof a composition according to claim 1.

31. A method for reducing erosion of an enamel surface comprising preparing an oral care composition according to claim 1, and applying the composition to the enamel surface.

32. A method for (i) reducing or inhibiting formation of dental caries, (ii) reducing, repairing or inhibiting pre-carious lesions of the enamel, (iii) reducing or inhibiting demineralization and promote remineralization of the teeth, (iv) reducing hypersensitivity of the teeth, (v) reducing or inhibiting gingivitis, (vi) promoting healing of sores or cuts in the mouth, (vii) reducing levels of acid producing bacteria, (viii) increasing relative levels of arginolytic bacteria, (ix) inhibiting microbial biofilm formation in the oral cavity, (x) raising and/or maintaining plaque pH at levels of at least pH 5.5 following sugar challenge, (xi) reducing plaque accumulation, (xii) treating, relieving or reducing dry mouth, (xiii) cleaning the teeth and oral cavity (xiv) reducing erosion, (xv) preventing stains and/or whitening teeth, (xvi) immunizing the teeth against cariogenic bacteria; and/or (xvii) promoting systemic health, including cardiovascular health; the method comprising applying a composition according to claim 1 to the oral cavity of a person in need thereof.
